(19) United States
(12) Patent Application Publication
(10) Pub. No.: US 2010/0041051 A1
(43) Pub. Date: Feb. 18, 2010

(54) Prostate Cancer Methylation Assay

(76) Inventors: Jonathan F. Baden, Bridgewater, NJ (US); George A. Green, IV, Newton, NJ (US); Jennifer Painter, Piscataway, NJ (US); Yixin Wang, Basking Ridge, NJ (US); Sean Wuxiong Cao, Three Bridges, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/536,326

(22) Filed: Aug. 5, 2009

Related U.S. Application Data
(60) Provisional application No. 61/086,218, filed on Aug. 5, 2008.

(51) Int. Cl. C12Q 1/68

(52) U.S. Cl. 435/6

(57) ABSTRACT

An assay for diagnosing or prognosticating prostate cancer incorporates the detection of hypermethylation of SEQ ID NO 1, SEQ ID NO 3, and SEQ ID NO 2 genes and may be incorporated into a nomogram.

This application claims the benefit of U.S Provisional Application No. 61/086,218 filed on Aug. 5, 2008.

### BACKGROUND

The measure of serum prostate-specific antigen (PSA) is currently the standard of care for prostate cancer screening. The low specificity of PSA tests has been shown to result in unnecessary biopsy of large numbers of patients and typically limits the PSA range that can be screened to patients with >4 ng/mL.

New assays use Methylation Specific PCR (MSP) to detect CpG island methylation (epigenetic modifications) within the promoter regions of three markers (GSTP1, RARβ2 and APC) that are indicative of the presence of prostate cancer. This assay has been evaluated with 337 post-DRE urine samples collected at 9 clinical sites (187 cancer & atypia/150 non-cancer). The patients ranged in age from 40-75 and had PSA levels from 2 to 10 ng/mL. A sensitivity of 52% and specificity of 81% was observed for the assay in the detection of prostate cancer as determined by histology on biopsy tissue. Through a logistic regression algorithm an area under the curve (AUC) value of 0.67 was adduced for the assay. When the assay was used in conjunction with a nomogram or the PCPT risk calculator an increase in AUC (0.69 and 0.72) and demonstrated statistical significance (p=0.008 and 0.043) was adduced when compared to the nomogram or PCPT risk calculator alone. The positive predictive value of the assay increased when one (48%), two (60%), or three (71%) markers were positive in the same subject. When a subset of 180 post-DRE urine samples (103 cancer & atypia/non-cancer 77) was prepared in accordance with the optimized assay procedure, a sensitivity of 60% and specificity of 81% (AUC 0.72) was observed.

### SUMMARY OF THE INVENTION

The invention is directed to an assay for detecting the hypermethylation of genes relating to prostate cancer includes reagents for detecting the presence of GSTP1, APC, RARβ2 or combinations thereof.

In another aspect of the invention, the reagents include a primer, probe, or scorpion reagent selected from the group of primers, probes, and Scorpion reagents set forth in Table 1.

In another aspect of the invention the assay is used in conjunction with a nomogram for determine the diagnosis or prognosis of a suspected prostate cancer patient.

### DETAILED DESCRIPTION

Hypermethylation assays that include the detection of GSTP, APC, and RARβ2 markers are described in, for example, US Patent Publication 20080254455 which is incorporated herein by reference. These assays have now been improved and can be used in conjunction with other diagnostic and risk factor indicators.

In a study with a population that consisted of 337 apparent healthy men with no previous history of prostate cancer, urine samples were obtained from 9 different urological clinical sites. Urine samples (up to 40 mL) were collected following a defined DRE that consists of depressing the prostate surface 0.5 to 1.0 cm, and moving from base to the apex and from the lateral to the median line for a minimum of three strokes per lobe. The contents of the urine collection container were transferred into a 50 mL transport tube containing 800 μL 0.5M EDTA. The transport tubes were stored at 2-8° C. for up to three days post collection and were shipped overnight with standard ice packs. Upon receipt, transport tubes were either centrifuged immediately at 3000 g for 10 min at 4° C. or split into equal parts and subsequently centrifuged at 3000 g for 10 min at 4° C. Urine samples were split to aid in both sample preparation optimization and estimation of overall performance. Supernatant was discarded and the resultant pellet is washed with cold PBS. DNA was extracted using the Gentra Puregene Kit (Qiagen, Germany) and modified using the Epitect Kit (Qiagen, Germany) according to the package insert. All samples were eluted in 25 μL volume. 5 μL of modified DNA was analyzed using the prostate cancer methylation assay on the SmartCycler (Cepheid, Sunnyvale, Calif.).

Primer and Scorpion probes (Biosearch Technologies, Novato, Calif.) for three methylation markers (GSTP1, RARB, and APC) and internal control β-Actin were chosen for use in a two-step multiplexed MSP assay. The first step, Amplification, consisted of 5 μL amplification mix, 5 μl enzyme mix and 5 μL sample added to a SmartCap tube (Cepheid, Sunnyvale, Calif.). The Enzyme Mix wass formulated for use in both the Amplification and Detection steps and consists of 8 mM Tris-HCl pH 8.0, 5 mM KCl, 0.005% BSA, 0.6U/μL FastStart Taq DNA polymerase and 0.016% ProClin® 300.

The Amplification step cycles were as follows: 95° C. for 5 min, followed by 18 cycles at 95° C. for 20 s, 55° C. for 30 s, 70° C .for 30 s, and 70° C. for 5 min. The Amplification mix contains 8 primers at 20 nM each for GSTP1, RARB, APC, 16 nM for β-Actin, 75 mM D-Trehalose dehydrate, 0.1% Tween® 20 Solution 10%, 25 mM Tris-HCl pH 8.0 1M, 1.75 mM MgCl₂ Solution, 1% DMSO, 0.155 mM dNTP Mix, 0.016% ProClin® 300.

Upon completion of the Amplification step the SmartCap tubes were removed from the instrumentation. The second step, Detection, consisted of 5 μL detection mix and 5 μl enzyme mix added to a SmartCap tube. The assay cycles as follows: 95° C. for 5 min, followed by 40 cycles of 95° C. for 20 s and 55° C. for 30 s. The detection mix was formulated exactly as described for the Amplification mix above with the following exception, 4 primers at 200 nM each for GSTP1, RARB, APC and β-Actin and 4 Scorpion probes at 200 nM each for GSTP1, RARB, APC and β-Actin instead of 8 primers. In each run, Negative (β-Actin) and Positive (GSTP1, APC, RARβ2) synthetic external controls were utilized to determine assay validity.

Classification analysis was based on the known biopsy results of the patients in the study population. Cycle threshold (Ct) values were used to generate independent assay cutoffs for the GSPT 1, RARβ2 and APC markers. To determine the No Test Rate (NTR) as a cause of insufficient DNA amount, a Ct value cutoff for β-Actin was used. A sample was considered positive for methylation if one Ct value from the set of 3 methylation markers was below the defined cutoff. Samples with Ct values above the defined cutoffs were scored as negative for methylation. NTR was calculated based on the Ct cutoff for β-Actin. Area Under the operating receiver Curve (AUC) values was calculated based on Receiver Operating Characteristic (ROC) analysis. AUC values for single-marker and multiple marker analysis were generated using MedCalc (MedCalc Software, Belgium). Logistic regression models were created using MedCalc for multiple marker analysis.

This assay was evaluated for its ability to discriminate prostate cancer patients from patients with a negative biopsy. The GSTP1, RARβ2, and APC Ct values in men with negative and positive biopsies were significantly different (p=0.009, 0.000 and 0.039 respectively) and demonstrated positive ROC curves . Combining the 3 markers, the assay demonstrated a sensitivity of 52% and specificity of 81% for detection of prostate cancer as determined by the histologic findings on biopsy tissue, (84 cancer and 104 non-cancer correctly called). Many of the false positives in the assay had an abnormal DRE and/or multiple markers that were positive. Among the potential explanation for the false positives is sampling error at the time of prostate biopsy, or the presence of methylated but non-cancerous prostate cells. A logistic regression algorithm using all 3 markers resulted in an AUC value of 0.67. Total serum PSA is commonly utilized as a risk factor to determine who should undergo prostate biopsy. The performance of PSA and the prostate cancer methylation assay were compared. ROC curve analysis of PSA demonstrated an AUC of 0.55 in this study population while this assay demonstrated statistical significance (p=0.01) when compared to PSA alone. More importantly, by both univariable and multivariable logistic regression models this assay was a significant predictor of prostate cancer (p=0.001) even when multiple risk factors were analyzed.

A combination of multiple risk factors in nomograms or predicative algorithms, rather than PSA alone is a growing trend within the published literature to provide greater efficacy and efficiency. A comparison of the prostate cancer methylation assay and a commonly used nomogram consisting of PSA, DRE result and age of patient and the PCPT risk calculator is shown. Information on the PCPT risk calculator parameters was obtained from 253 subjects. A logistic regression algorithm using the nomogram resulted in AUC value of 0.61. Interestingly, the PCPT risk calculator resulted in an AUC of 0.67. The prostate cancer methylation assay was not statistical significant (p=0. 150 and 0.935, respectively) when compared to nomogram or PCPT risk calculator. However, this assay in conjunction with the nomogram or the PCPT risk calculator improved the AUC (0.69 and 0.72, respectively) and demonstrated statistical significance (p=0.008 and 0.043, respectively) when compared to the nomogram or PCPT calculator alone. To further assess the prostate cancer methylation assay data was evaluated from individual clinical sites. The difference between sites and the overall population tested was not significant when an independent analysis of ROC curves was performed.

The predictive value of this assay is underscored by the high specificity of the GSTP1, RARβ2 and APC markers. When the patient cohort was stratified according to having 1, 2, or 3 markers positive, the positive predictive value (PPV) of the assay performance improved (48%-71%). This suggests that there is a higher likelihood of having cancer when 2 or more markers are present in the assay.

The predicative value of the prostate cell methylation assay is emphasized by the high specificity of the assay. This can be attributed to the MSP methodology employed in comparison to expression-based assays. The markers of this assay demonstrated high specificity, 90%, 89% and 95% respectively. Another advantage of this assay over the PCA3 marker is the unique nature of the 3 gene multiplex assay that enables the clinician to have a higher level of confidence when a patient presents with multiple markers. The observed PPV of this assay at 25% cancer prevalence improved when one (48%), two (60%), or three (71%) markers were positive in the same subjects.

The algorithm used to provide an assay score is based on a logistic function of the linear combination of methylation specific PCR (MSP) Ct values and will be associated with the probability of positive biopsy. The model places individuals at high or low risk values, where decisions are more easily made. Specifically, “high” scores (>60.00) will have likelihood ratios >3.0 and “low” scores (<29.00) will have likelihood ratios <0.35. The score allows for the patient to have a more informed discussion with his doctor concerning the probability of having a positive biopsy.

Score=100×1/[1+exp(Linear Ct Combination)],

where “Linear Ct Combination” is formed based on the trial data:

1.7887+(−0.0686×GSTP1_Ct)+(−0.03947×RARβ2_Ct)+(−0.01263×APC_Ct)+(0.09862×β-actin_Ct)

Assay score when combined with other known risk factors will be a statistically significant factor in predicting a positive prostate biopsy. The risk factors will include age, family history of prostate cancer, PSA level, race, and previous negative prostate biopsy.

Designs in table 1 show improved specificity as compared to original feasibility designs when markers were evaluated on CpGM and CpGU DNA. The larger the difference in Ct value is from CpGM in comparison to CpGU the greater specificity of the marker design.

TABLE 1

Primers and Scorpion ™ probes sequences (for 3 methylation markers (GSTP1,
RARβ2, and APC) and internal control (β-actin)

Sequence ID

Amplification Mix

| | |
|---|---|
| Forward Primers | |
| TTTTTGCGGTCGACGTTCG | GSTP1-F |
| GATATAAGGTTAGGGATAGGATAG | β-Actin-F |
| CCTATACCCCACTACGAAATACGA | APC-F |
| GGGGATTAGAATTTTTTTATGCG | RARβ2-F |
| Reverse Primers | |
| CGCCCCAATACTAAATCACG | GSTP1-R |
| AACACACAATAACAAACACAAATTCAC | β-Actin-R |
| GTCGGTTACGTGCGTTTATATTTAG | APC-R |
| CTTACAAAAAACCTTCCGAATACG | RARβ2-R |

Detection Mix

| | |
|---|---|
| Scorpion Probe/Primer Hybrid | |
| FAM-CCGGGCGAACTCCCGCCGAGCCCGG-BHQ-HEG-TCGGGGTGTAGCGGTCGTCG | GSTP1-FAM |
| Q670-CCGGGGCCTCCATCACCACCCCGG-BHQ2-HEG-TATAGGTTGGGGAAGTTTGTTTTTG | β-Actin-Q670 |
| TR-GCCGGCGGGTTTTCGACGGGCCGGC-BHQ2-HEG-CGAACCAAAACGCTCCCCA | APC-TxR |
| Q570-CGCGGGCTACCCCGACGATACCCGCG-BHQ2-HEG-GGGATGTCGAGAACGCGAGCGA | RARβ2-Q570 |
| Reverse Primers | |
| CGCCCCAATACTAAATCACG | GSTP1-R |
| AACACACAATAACAAACACAAATTCAC | β-Actin-R |
| GTCGGTTACGTGCGTTTATATTTAG | APC-R |
| CTTACAAAAAACCTTCCGAATACG | RARβ2-R |

Improper folding of original GSTP1 scorpion design can act as a substrate for taq cleavage, this leads to degradation of the quencher molecule that causes a steady drift in background as compared to new GSTP1 design. New designs improve overall performance.

1. A kit for detecting the hypermethylation of genes relating to prostate cancer comprising reagents for detecting the presence of SEQ ID NO 1, SEQ ID NO 3, SEQ ID NO 2 or combinations thereof wherein said reagents include a primer, probe, or scorpion reagent selected from the group of primers, probes, and scrorpion reagents set forth in Table 1.

2. A method of diagnosing or prognosticating prostate cancer comprising detecting the hypermethylation of SEQ ID NO 1, SEQ ID NO 3, SEQ ID NO 2 genes or combinations thereof with reagents that include a primer, probe, or scorpion reagent selected from the group of primers, probes, and scrorpion reagents set forth in Table 1.

3. The method of claim 2 wherein the analysis of hypermethylation is used in conjunction with other risk factors or indicators of prostate cancer diagnosis or prognosis.

4. The method of claim 3 wherein other risk factors are included in a nomogram.


SEQUENCE LISTING (16 sequences)

SEQ ID NO: 1 - DNA, 4260 bp, Homo sapiens
aacaagagatcaatatctagaataaatggagatctgcaaatcaacagaaagtaggcagcaaagccaaagaaaatagcctaaggcacagccactaaaaggaacgtgatcatgtcctttgcagggacatgggtggagctggaagccgttagcctcagcaaactcacacaggaacagaaaaccagcgagaccgcatggtctcacttataagtgggagctgaacaatgagaacacatggtcacatggcggcgatcaacacacactggtgcctgttgagcggggtgctggggagggagagtaccaggaagaatagctaagggatactgggcttaatacctgggtgatgggatgatctgtacagcaaaccatcatggcgcacacacctatgtaacaaacctgcacatcctgcacatgtaccccagaacttcaaataaaagttggacggccaggcgtggtggctcacgcctgtaatcccagcactttgggaagccgaggcgtgcagatcacctaaggtcaggagttcgagaccagcccggccaacatggtgaaaccccgtctctactaaaaatacaaaaatcagccagatgtggcacgcacctataattccacctactcgggaggctgaagcagaattgcttgaacccgagaggcggaggttgcagtgagccgccgagatcgcgccactgcactccagcctgggccacagcgtgagactacgtcataaaataaaataaaataacacaaaataaaataaaataaaataaaataaaataaaataataaaataaaataaaataaaataaaataaaataaaataaagcaatttcctttcctctaagcggcctccacccctctcccctgccctgtgaagcgggtgtgcaagctccgggatcgcagcggtcttagggaatttccccccgcgatgtcccggcgcgccagttcgctgcgcacacttcgctgcggtcctcttcctgctgtctgtttactccctaggccccgctggggacctgggaaagagggaaaggcttccccggccagctgcgcggcgactccggggactccagggcgcccctctgcggccgacgcccggggtgcagcggccgccggggctggggccggcgggagtccgcgggaccctccagaagagcggccggcgccgtgactcagcactggggcggagcggggcgggaccacccttataaggctcggaggccgcgaggccttcgctggagtttcgccgccgcagtcttcgccaccagtgagtacgcgcggcccgctccccggggatggggctcagagctcccagcatggggccaacccgcagcatcaggcccgggctcccggcagggctcctcgcccacctcgagacccgggacgggggcctaggggacccaggacgtccccagtgccgttagcggctttcagggggcccggagcgcctcggggagggatgggaccccgggggcggggagggggggcaggctgcgctcaccgcgccttggcatcctcccccgggctccagcaaacttttctttgttcgctgcagtgccgccctacaccgtggtctatttcccagttcgaggtaggagcatgtgtctggcagggaagggaggcaggggctggggctgcagcccacagcccctcgcccacccggagagatccgaacccccttatccctccgtcgtgtggcttttaccccgggcctccttcctgttccccgcctctcccgccatgcctgctccccgccccagtgttgtgtgaaatcttcggaggaacctgtttacctgttccctccctgcactcctgacccctccccgggttgctgcgaggcggagtcggcccggtccccacatctcgtacttctccctccccgcaggccgctgcgcggccctgcgcatgctgctggcagatcagggccagagctggaaggaggaggtggtgaccgtggagacgtggcaggagggctcactcaaagcctcctgcgtaagtgaccatgcccgggcaaggggagggggtgctgggccttagggggctgtgactaggatcgggggacgcccaagctcagtgcccctccctgagccatgcctcccccaacagctatacgggcagctccccaagttccaggacggagacctcaccctgtaccagtccaataccatcctgcgtcacctgggccgcacccttggtgagtcttgaacctccaagtccagggcaggcatgggcaagcctctgcccccggagcccttttgtttaaatcagctgccccgcagccctctggagtggaggaaactgagacccactgaggttacgtagtttgcccaaggtcaagcctgggtgcctgcaatccttgccctgtgccaggctgcctcccaggtgtcaggtgagctctgagcacctgctgtgtggcagtctctcatccttccacgcacatcctcttcccctcctcccaggctggggctcacagacagccccctggttggcccatccccagtgactgtgtgttgatcaggcgcccagtcacgcggcctgctcccctccacccaaccccagggctctatgggaaggaccagcaggaggcagccctggtggacatggtgaatgacggcgtggaggacctccgctgcaaatacatctccctcatctacaccaactatgtgagcatctgcaccagggttgggcactgggggctgaacaaagaaaggggcttcttgtgccctcaccccccttacccctcaggtggcttgggctgaccccttcttgggtcagggtgcaggggctgggtcagctctgggccaggggcccaggggcctgggacaagacacaacctgcacccttattgcctgggacatcaaccagccaagtaacgggtcatgggggcgagtgcaaggacagagacctccagcaactggtggtttctgatctcctggggtggcgagggcttcctggagtagccagaggtggaggaggatttgtcgccagtttctggatggaggtgctggcacttttagctgaggaaaatatgcagacacagagcacatttggggacctgggaccagttcagcagaggcagcgtgtgtgcgcgtgcgtgtgcgtgtgtgtgcgtgtgtgtgtgtacgcttgcatttgtgtcgggtgggtaaggagatagagatgggcgggcagtaggcccaggtcccgaaggccttgaacccactggtttggagtctcctaagggcaatgggggccattgagaagtctgaacagggctgtgtctgaatgtgaggtctagaaggatcctccagagaagccagctctaaagcttttgcaatcatctggtgagagaacccagcaaggatggacaggcagaatggaatagagatgagttggcagctgaagtggacaggatttggtactagcctggttgtggggagcaagcagaggagaatctgggactctggtgtctggcctggggcagacgggggtgtctcaggggctgggagggatgagagtaggatgatacatggtggtgtctggcaggaggcgggcaaggatgactatgtgaaggcactgcccgggcaactgaagccttttgagaccctgctgtcccagaaccagggaggcaagaccttcattgtgggagaccaggtgagcatctggccccatgctgttccttcctcgccaccctctgcttccagatggacacaggtgtgagccatttgtttagcaaagcagagcagacctaggggatgggcttaggccctctgcccccaattcctccagcctgctcccgctggctgagtccctagcccccctgccctgcagatctccttcgctgactacaacctgctggacttgctgctgatccatgaggtcctagcccctggctgcctggatgcgttccccctgctctcagcatatgtggggcgcctcagtgcccggcccaagctcaaggccttcctggcctcccctgagtacgtgaacctccccatcaatggcaacgggaaacagtgagggttggggggactctgagcgggaggcagagtttgccttcctttctccaggaccaataaaatttctaagagagctactatgagcactgtgtttcctgggacggggcttaggggttctcagcctcgaggtcggtgggagggcagagcagaggactagaaaacagctcctccagcacagtcagtggcttcctggagccctcagcctggctgtgtttactgaacctcacaaactagaagaggaagaaaaaaaaagagagagagaaacaaagagaaat

SEQ ID NO: 2 - DNA, 2764 bp, Homo sapiens
gtgacagaagtagtaggaagtgagctgttcagaggcaggagggtctattctttgccaaaggggggaccagaattccccatgcgagctgtttgaggactgggatgccgagaacgcgagcgatccgagcagggtttgtctgggcaccgtcggggtaggatccggaacgcattcggaaggctttttgcaagcatttacttggaaggagaacttgggatctttctgggaaccccccgccccggctggattggccgagcaagcctggaaaatgcaattgaaacacagagcaccagctctgaggaactcgtcccaagccccccatctccacttcctccccctcgagtgtacaaaccctgcttcgtctgccaggacaaatcatcagggtaccactatggggtcagcgcctgtgagggatgtaagggctttttccgcagaagtattcagaagaatatgatttacacttgtcaccgagataagaactgtgttattaataaagtcaccaggaatcgatgccaatactgtcgactccagaagtgctttgaagtgggaatgtccaaagaatctgtcaggaatgacaggaacaagaaaaagaaggagacttcgaagcaagaatgcacagagagctatgaaatgacagctgagttggacgatctcacagagaagatccgaaaagctcaccaggaaactttcccttcactctgccagctgggtaaatacaccacgaattccagtgctgaccatcgagtccgactggacctgggcctctgggacaaattcagtgaactggccaccaagtgcattattaagatcgtggagtttgctaaacgtctgcctggtttcactggcttgaccatcgcagaccaaattaccctgctgaaggccgcctgcctggacatcctgattcttagaatttgcaccaggtataccccagaacaagacaccatgactttctcagacggccttaccctaaatcgaactcagatgcacaatgctggatttggtcctctgactgaccttgtgttcacctttgccaaccagctcctgcctttggaaatggatgacacagaaacaggccttctcagtgccatctgcttaatctgtggagaccgccaggaccttgaggaaccgacaaaagtagataagctacaagaaccattgctggaagcactaaaaatttatatcagaaaaagacgacccagcaagcctcacatgtttccaaagatcttaatgaaaatcacagatctccgtagcatcagtgctaaaggtgcagagcgtgtaattaccttgaaaatggaaattcctggatcaatgccacctctcattcaagaaatgctggagaattctgaaggacatgaacccttgaccccaagttcaagtgggaacacagcagagcacagtcctagcatctcacccagctcagtggaaaacagtggggtcagtcagtcaccactcgtgcaataagacattttctagctacttcaaacattccccagtaccttcagttccaggatttaaaatgcaagaaaaaacatttttactgctgcttagtttttggactgaaaagatattaaaactcaagaaggaccaagaagttttcatatgtatcaatatatatactcctcactgtgtaacttacctagaaatacaaacttttccaattttaaaaaatcagccatttcatgcaaccagaaactagttaaaagcttctattttcctctttgaacactcaagatgcatggcaaagacccagtcaaaatgatttacccctggttaagtttctgaagactttgtacatacagaagtatggctctgttctttctatactgtatgtttggtgctttccttttgtcttgcatactcaaaataaccatgacaccaaggttatgaaatagactactgtacacgtctacctaggttcaaaaagataactgtcttgctttcatggaatagtcaagacatcaaggtaaggaaacaggactattgacaggactattgtacagtatgacaagataaggctgaagatattctactttagttagtatggaagcttgtctttgctctttctgatgctctcaaactgcatcttttatttcatgttgcccagtaaaagtatacaaattccctgcactagcagaagagaattctgtatcagtgtaactgccagttcagttaatcaaatgtcatttgttcaattgttaatgtcactttaaattaaaagtggtttattacttgtttaatgacataactacacagttagttaaaaaaaatttttttacagtaatgatagcctccaaggcagaaacacttttcagtgttaagtttttgtttacttgttcacaagccattagggaaatttcatgggataattagcaggctggtctaccactggaccatgtaactctagtgtccttcctgattcatgcctgatattgggatttttttccagcccttcttgatgccaagggctaattatattacatcccaaagaaacaggcatagaatctgcctcctttgaccttgttcaatcactatgaagcagagtgaaagctgtggtagagtggttaacagatacaagtgtcagtttcttagttctcatttaagcactactggaatttttttttttgatatattagcaagtctgtgatgtactttcactggctctgtttgtacattgagattgtttgtttaacaatgctttctatgttcatatactgtttacctttttccatggactctcctggcaaagaataaaatatatttatttttt

SEQ ID NO: 3 - DNA, 10386 bp, Homo sapiens
  misc_feature (9521)..(9521): n is a, c, g, or t
attgaggactcggaaatgaggtccaagggtagccaaggatggctgcagcttcatatgatcagttgttaaagcaagttgaggcactgaagatggagaactcaaatcttcgacaagagctagaagataattccaatcatcttacaaaactggaaactgaggcatctaatatgaaggaagtacttaaacaactacaaggaagtattgaagatgaagctatggcttcttctggacagattgatttattagagcgtcttaaagagcttaacttagatagcagtaatttccctggagtaaaactgcggtcaaaaatgtccctccgttcttatggaagccgggaaggatctgtatcaagccgttctggagagtgcagtcctgttcctatgggttcatttccaagaagagggtttgtaaatggaagcagagaaagtactggatatttagaagaacttgagaaagagaggtcattgcttcttgctgatcttgacaaagaagaaaaggaaaaagactggtattacgctcaacttcagaatctcactaaaagaatagatagtcttcctttaactgaaaatttttccttacaaacagatatgaccagaaggcaattggaatatgaagcaaggcaaatcagagttgcgatggaagaacaactaggtacctgccaggatatggaaaaacgagcacagcgaagaatagccagaattcagcaaatcgaaaaggacatacttcgtatacgacagcttttacagtcccaagcaacagaagcagagaggtcatctcagaacaagcatgaaaccggctcacatgatgctgagcggcagaatgaaggtcaaggagtgggagaaatcaacatggcaacttctggtaatggtcagggttcaactacacgaatggaccatgaaacagccagtgttttgagttctagtagcacacactctgcacctcgaaggctgacaagtcatctgggaaccaaggtggaaatggtgtattcattgttgtcaatgcttggtactcatgataaggatgatatgtcgcgaactttgctagctatgtctagctcccaagacagctgtatatccatgcgacagtctggatgtcttcctctcctcatccagcttttacatggcaatgacaaagactctgtattgttgggaaattcccggggcagtaaagaggctcgggccagggccagtgcagcactccacaacatcattcactcacagcctgatgacaagagaggcaggcgtgaaatccgagtccttcatcttttggaacagatacgcgcttactgtgaaacctgttgggagtggcaggaagctcatgaaccaggcatggaccaggacaaaaatccaatgccagctcctgttgaacatcagatctgtcctgctgtgtgtgttctaatgaaactttcatttgatgaagagcatagacatgcaatgaatgaactagggggactacaggccattgcagaattattgcaagtggactgtgaaatgtacgggcttactaatgaccactacagtattacactaagacgatatgctggaatggctttgacaaacttgacttttggagatgtagccaacaaggctacgctatgctctatgaaaggctgcatgagagcacttgtggcccaactaaaatctgaaagtgaagacttacagcaggttattgcaagtgttttgaggaatttgtcttggcgagcagatgtaaatagtaaaaagacgttgcgagaagttggaagtgtgaaagcattgatggaatgtgctttagaagttaaaaaggaatcaaccctcaaaagcgtattgagtgccttatggaatttgtcagcacattgcactgagaataaagctgatatatgtgctgtagatggtgcacttgcatttttggttggcactcttacttaccggagccagacaaacactttagccattattgaaagtggaggtgggatattacggaatgtgtccagcttgatagctacaaatgaggaccacaggcaaatcctaagagagaacaactgtctacaaactttattacaacacttaaaatctcatagtttgacaatagtcagtaatgcatgtggaactttgtggaatctctcagcaagaaatcctaaagaccaggaagcattatgggacatgggggcagttagcatgctcaagaacctcattcattcaaagcacaaaatgattgctatgggaagtgctgcagctttaaggaatctcatggcaaataggcctgcgaagtacaaggatgccaatattatgtctcctggctcaagcttgccatctcttcatgttaggaaacaaaaagccctagaagcagaattagatgctcagcacttatcagaaacttttgacaatatagacaatttaagtcccaaggcatctcatcgtagtaagcagagacacaagcaaagtctctatggtgattatgtttttgacaccaatcgacatgatgataataggtcagacaattttaatactggcaacatgactgtcctttcaccatatttgaatactacagtgttacccagctcctcttcatcaagaggaagcttagatagttctcgttctgaaaaagatagaagtttggagagagaacgcggaattggtctaggcaactaccatccagcaacagaaaatccaggaacttcttcaaagcgaggtttgcagatctccaccactgcagcccagattgccaaagtcatggaagaagtgtcagccattcatacctctcaggaagacagaagttctgggtctaccactgaattacattgtgtgacagatgagagaaatgcacttagaagaagctctgctgcccatacacattcaaacacttacaatttcactaagtcggaaaattcaaataggacatgttctatgccttatgccaaattagaatacaagagatcttcaaatgatagtttaaatagtgtcagtagtagtgatggttatggtaaaagaggtcaaatgaaaccctcgattgaatcctattctgaagatgatgaaagtaagttttgcagttatggtcaatacccagccgacctagcccataaaatacatagtgcaaatcatatggatgataatgatggagaactagatacaccaataaattatagtcttaaatattcagatgagcagttgaactctggaaggcaaagtccttcacagaatgaaagatgggcaagacccaaacacataatagaagatgaaataaaacaaagtgagcaaagacaatcaaggaatcaaagtacaacttatcctgtttatactgagagcactgatgataaacacctcaagttccaaccacattttggacagcaggaatgtgtttctccatacaggtcacggggagccaatggttcagaaacaaatcgagtgggttctaatcatggaattaatcaaaatgtaagccagtctttgtgtcaagaagatgactatgaagatgataagcctaccaattatagtgaacgttactctgaagaagaacagcatgaagaagaagagagaccaacaaattatagcataaaatataatgaagagaaacgtcatgtggatcagcctattgattatagtttaaaatatgccacagatattccttcatcacagaaacagtcattttcattctcaaagagttcatctggacaaagcagtaaaaccgaacatatgtcttcaagcagtgagaatacgtccacaccttcatctaatgccaagaggcagaatcagctccatccaagttctgcacagagtagaagtggtcagcctcaaaaggctgccacttgcaaagtttcttctattaaccaagaaacaatacagacttattgtgtagaagatactccaatatgtttttcaagatgtagttcattatcatctttgtcatcagctgaagatgaaataggatgtaatcagacgacacaggaagcagattctgctaataccctgcaaatagcagaaataaaagaaaagattggaactaggtcagctgaagatcctgtgagcgaagttccagcagtgtcacagcaccctagaaccaaatccagcagactgcagggttctagtttatcttcagaatcagccaggcacaaagctgttgaattttcttcaggagcgaaatctccctccaaaagtggtgctcagacacccaaaagtccacctgaacactatgttcaggagaccccactcatgtttagcagatgtacttctgtcagttcacttgatagttttgagagtcgttcgattgccagctccgttcagagtgaaccatgcagtggaatggtaagtggcattataagccccagtgatcttccagatagccctggacaaaccatgccaccaagcagaagtaaaacacctccaccacctcctcaaacagctcaaaccaagcgagaagtacctaaaaataaagcacctactgctgaaaagagagagagtggacctaagcaagctgcagtaaatgctgcagttcagagggtccaggttcttccagatgctgatactttattacattttgccacggaaagtactccagatggattttcttgttcatccagcctgagtgctctgagcctcgatgagccatttatacagaaagatgtggaattaagaataatgcctccagttcaggaaaatgacaatgggaatgaaacagaatcagagcagcctaaagaatcaaatgaaaaccaagagaaagaggcagaaaaaactattgattctgaaaaggacctattagatgattcagatgatgatgatattgaaatactagaagaatgtattatttctgccatgccaacaaagtcatcacgtaaagcaaaaaagccagcccagactgcttcaaaattacctccacctgtggcaaggaaaccaagtcagctgcctgtgtacaaacttctaccatcacaaaacaggttgcaaccccaaaagcatgttagttttacaccgggggatgatatgccacgggtgtattgtgttgaagggacacctataaacttttccacagctacatctctaagtgatctaacaatcgaatcccctccaaatgagttagctgctggagaaggagttagaggaggagcacagtcaggtgaatttgaaaaacgagataccattcctacagaaggcagaagtacagatgaggctcaaggaggaaaaacctcatctgtaaccatacctgaattggatgacaataaagcagaggaaggtgatattcttgcagaatgcattaattctgctatgcccaaagggaaaagtcacaagcctttccgtgtgaaaaagataatggaccaggtccagcaagcatctgcgtcgtcttctgcacccaacaaaaatcagttagatggtaagaaaaagaaaccaacttcaccagtaaaacctataccacaaaatactgaatataggacacgtgtaagaaaaaatgcagactcaaaaaataatttaaatgctgagagagttttctcagacaacaaagattcaaagaaacagaatttgaaaaataattccaaggacttcaatgataagctcccaaataatgaagatagagtcagaggaagttttgcttttgattcacctcatcattacacgcctattgaaggaactccttactgtttttcacgaaatgattctttgagttctctagattttgatgatgatgatgttgacctttccagggaaaaggctgaattaagaaaggcaaaagaaaataaggaatcagaggctaaagttaccagccacacagaactaacctccaaccaacaatcagctaataagacacaagctattgcaaagcagccaataaatcgaggtcagcctaaacccatacttcagaaacaatccacttttccccagtcatccaaagacataccagacagaggggcagcaactgatgaaaagttacagaattttgctattgaaaatactccagtttgcttttctcataattcctctctgagttctctcagtgacattgaccaagaaaacaacaataaagaaaatgaacctatcaaagagactgagccccctgactcacagggagaaccaagtaaacctcaagcatcaggctatgctcctaaatcatttcatgttgaagataccccagtttgtttctcaagaaacagttctctcagttctcttagtattgactctgaagatgacctgttgcaggaatgtataagctccgcaatgccaaaaaagaaaaagccttcaagactcaagggtgataatgaaaaacatagtcccagaaatatgggtggcatattaggtgaagatctgacacttgatttgaaagatatacagagaccagattcagaacatggtctatcccctgattcagaaaattttgattggaaagctattcaggaaggtgcaaattccatagtaagtagtttacatcaagctgctgctgctgcatgtttatctagacaagcttcgtctgattcagattccatcctttccctgaaatcaggaatctctctgggatcaccatttcatcttacacctgatcaagaagaaaaaccctttacaagtaataaaggcccacgaattctaaaaccaggggagaaaagtacattggaaactaaaaagatagaatctgaaagtaaaggaatcaaaggaggaaaaaaagtttataaaagtttgattactggaaaagttcgatctaattcagaaatttcaggccaaatgaaacagccccttcaagcaaacatgccttcaatctctcgaggcaggacaatgattcatattccaggagttcgaaatagctcctcaagtacaagtcctgtttctaaaaaaggcccaccccttaagactccagcctccaaaagccctagtgaaggtcaaacagccaccacttctcctagaggagccaagccatctgtgaaatcagaattaagccctgttgccaggcagacatcccaaataggtgggtcaagtaaagcaccttctagatcaggatctagagattcgaccccttcaagacctgcccagcaaccattaagtagacctatacagtctcctggccgaaactcaatttcccctggtagaaatggaataagtcctcctaacaaattatctcaacttccaaggacatcatcccctagtactgcttcaactaagtcctcaggttctggaaaaatgtcatatacatctccaggtagacagatgagccaacagaaccttaccaaacaaacaggtttatccaagaatgccagtagtattccaagaagtgagtctgcctccaaaggactaaatcagatgaataatggtaatggagccaataaaaaggtagaactttctagaatgtcttcaactaaatcaagtggaagtgaatctgatagatcagaaagacctgtattagtacgccagtcaactttcatcaaagaagctccaagcccaaccttaagaagaaaattggaggaatctgcttcatttgaatctctttctccatcatctagaccagcttctcccactaggtcccaggcacaaactccagttttaagtccttcccttcctgatatgtctctatccacacattcgtctgttcaggctggtggatggcgaaaactcccacctaatctcagtcccactatagagtataatgatggaagaccagcaaagcgccatgatattgcacggtctcattctgaaagtccttctagacttccaatcaataggtcaggaacctggaaacgtgagcacagcaaacattcatcatcccttcctcgagtaagcacttggagaagaactggaagttcatcttcaattctttctgcttcatcagaatccagtgaaaaagcaaaaagtgaggatgaaaaacatgtgaactctatttcaggaaccaaacaaagtaaagaaaaccaagtatccgcaaaaggaacatggagaaaaataaaagaaaatgaattttctcccacaaatagtacttctcagaccgtttcctcaggtgctacaaatggtgctgaatcaaagactctaatttatcaaatggcacctgctgtttctaaaacagaggatgtttgggtgagaattgaggactgtcccattaacaatcctagatctggaagatctcccacaggtaatactcccccggtgattgacagtgtttcagaaaaggcaaatccaaacattaaagattcaaaagataatcaggcaaaacaaaatgtgggtaatggcagtgttcccatgcgtaccgtgggtttggaaaatcgcctgaactcctttattcaggtggatgcccctgaccaaaaaggaactgagataaaaccaggacaaaataatcctgtccctgtatcagagactaatgaaagttctatagtggaacgtaccccattcagttctagcagctcaagcaaacacagttcacctagtgggactgttgctgccagagtgactccttttaattacaacccaagccctaggaaaagcagcgcagatagcacttcagctcggccatctcagatcccaactccagtgaataacaacacaaagaagcgagattccaaaactgacagcacagaatccagtggaacccaaagtcctaagcgccattctgggtcttaccttgtgacatctgtttaaaagagaggaagaatgaaactaagaaaattctatgttaattacaactgctatatagacattttgtttcaaatgaaactttaaaagactgaaaaattttgtaaataggtttgattcttgttagagggtttttgttctggaagccatatttgatagtatactttgtcttcactggtcttattttgggaggcactcttgatggttaggaaaaaaatagtaaagccaagtatgtttgtacagtatgttttacatgtatttaaagtagcatcccatcccaacttcctttaattattgcttgtcttaaaataatgaacactacagatagaaaatatgatatattgctgttatcaatcatttctagattataaactgactaaacttacatcagggaaaaattggtatttatgcaaaaaaaaatgtttttgtccttgtgagtccatctaacatcataattaatcatgtggctgtgaaattcacagtaatatggttcccgatgaacaagctttacccagcctgtttgctttactgcatgaatgaaactgatggttcaatttcagaagtaatgattaacagttatgtggtcacatgatgtgcatagagatagctacagtgtaataatttacactattttgtgctccaaacaaaacaaaaatctgtgtaactgtaaaacattgaatgaaactattttacctgaactagattttatctgaaagtaggtagaatttttgctatgctgtaatttgttgtatattctggtatttgaggtgagatggctgctcttttattaatgagacatgaattgtgtctcaacagaaactaaatgaacatttcagaataaattattgctgtatgtaaactgttactgaaattggtatttgtttgaagggtcttgtttcacatttgtattaataattgtttaaaatgcctcttttaaaagcttatataaattttttncttcagcttctatgcattaagagtaaaattcctcttactgtaataaaaacaattgaagaagactgttgccacttaaccattccatgcgttggcacttatctattcctgaaattcttttatgtgattagctcatcttgatttttaacatttttccacttaaacttttttttcttactccactggagctcagtaaaagtaaattcatgtaatagcaatgcaagcagcctagcacagactaagcattgagcataataggcccacataatttcctctttcttaatattatagaaattctgtacttgaaattgattcttagacattgcagtctcttcgaggctttacagtgtaaactgtcttgccccttcatcttcttgttgcaactgggtctgacatgaacactttttatcaccctgtatgttagggcaagatctcagcagtgaagtataatcagcactttgccatgctcagaaaattcaaatcacatggaactttagaggtagatttaatacgattaagatattcagaagtatattttagaatccctgcctgttaaggaaactttatttgtggtaggtacagttctggggtacatgttaagtgtccccttatacagtggagggaagtcttccttcctgaaggaaaataaactgacacttattaactaagataatttacttaatatatcttccctgatttgttttaaaagatcagagggtgactgatgatacatgcatacatatttgttgaataaatgaaaatttatttttagtgataagattcatacactctgtatttggggagagaaaacctttttaagcatggtggggcactcagataggagtgaatacacctacctggtggtcat

SEQ ID NO: 4 - DNA, 1792 bp, Homo sapiens
cgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcattgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttccccctttttgtcccccaacttgagatgtatgaaggcttttggtctccctgggagtgggtggaggcagccagggcttacctgtacactgacttgagaccagttgaataaaagtgcacacctt

SEQ ID NO: 5 - DNA, 19 bp, Homo sapiens
  misc_feature: GSTP1-F
tttttgcggtcgacgttcg

SEQ ID NO: 6 - DNA, 24 bp, Homo sapiens
  misc_feature: beta-Actin-F
gatataaggttagggataggatag

SEQ ID NO: 7 - DNA, 24 bp, Homo sapiens
  misc_feature: APC-F
cctataccccactacgaaatacga

SEQ ID NO: 8 - DNA, 23 bp, Homo sapiens
  misc_feature: RARB2-F
ggggattagaatttttttatgcg

SEQ ID NO: 9 - DNA, 20 bp, Homo sapiens
  misc_feature: GSTP1-R
cgccccaatactaaatcacg

SEQ ID NO: 10 - DNA, 27 bp, Homo sapiens
  misc_feature: beta-Actin-R
aacacacaataacaaacacaaattcac

SEQ ID NO: 11 - DNA, 25 bp, Homo sapiens
  misc_feature: APC-R
gtcggttacgtgcgtttatatttag

SEQ ID NO: 12 - DNA, 24 bp, Homo sapiens
  misc_feature: RARB2-R
cttacaaaaaaccttccgaatacg

SEQ ID NO: 13 - DNA, 45 bp, Homo sapiens
  misc_feature: GSTP1-FAM
ccgggcgaactcccgccgagcccggtcggggtgtagcggtcgtcg

SEQ ID NO: 14 - DNA, 49 bp, Homo sapiens
  misc_feature: beta-Actin-Q670
ccggggcctccatcaccaccccggtataggttggggaagtttgtttttg

SEQ ID NO: 15 - DNA, 44 bp, Homo sapiens
  misc_feature: APC-TxR
gccggcgggttttcgacgggccggccgaaccaaaacgctcccca

SEQ ID NO: 16 - DNA, 48 bp, Homo sapiens
  misc_feature: RARB2-570
cgcgggctaccccgacgatacccgcggggatgtcgagaacgcgagcga